SEQUENCE LISTING (13 sequences)

SEQ ID NO: 1 - DNA, 40 bp, Artificial Sequence
  AB-40 mer - Single Stranded
ggaaccttggccttaaccaaccaaggttccggttaaggaa

SEQ ID NO: 2 - DNA, 41 bp, Artificial Sequence
  CD-41 mer - Single Stranded
gcaacgtgtgccgttaacgaacctaggatgggcattaggta

SEQ ID NO: 3 - DNA, 41 bp, Artificial Sequence
  B′C′-41 mer - Single Stranded
ttccttaaccggaaccttggttcgttaacggcacacgttgc

SEQ ID NO: 4 - DNA, 40 bp, Artificial Sequence
  D′A′-40 mer - Single Stranded
tacctaatgcccatcctaggttggttaaggccaaggttcc

SEQ ID NO: 5 - DNA, 40 bp, Artificial Sequence
  B′A′-40 mer - Single Stranded
ttccttaaccggaaccttggttggttaaggccaaggttcc

SEQ ID NO: 6 - DNA, 20 bp, Artificial Sequence
  B′-20 mer - Single Stranded
ttccttaaccggaaccttgg

SEQ ID NO: 7 - DNA, 20 bp, Artificial Sequence
  D-20 mer - Single Stranded
cctaggatgggcattaggta

SEQ ID NO: 8 - DNA, 35 bp, Artificial Sequence
  H′1N-35 mer - Double Stranded
ggccaaaaaagcattgcttatcaatttgttgcacc

SEQ ID NO: 9 - DNA, 35 bp, Artificial Sequence
  H′1N′-35 mer - Double Stranded
cggtgcaacaaattgataagcaatgcttttttggc

SEQ ID NO: 10 - DNA, 44 bp, Artificial Sequence
  Duplex 1 -44 mer - Double Stranded
tacgtttgttgcatgcttacaaattgttgcaacgttgttttacg

SEQ ID NO: 11 - DNA, 44 bp, Artificial Sequence
  Duplex 1′ -44 mer - Double Stranded
cgtaaaacaacgttgcttacaatttgttgcatgcaacaaacgta

SEQ ID NO: 12 - DNA, 36 bp, Artificial Sequence
  TT8AT - 36 mer - Double Stranded
cggtgcaacaatatgataagctttgcttttttggcc

SEQ ID NO: 13 - DNA, 35 bp, Artificial Sequence; identical to SEQ ID NO: 8
  H′1N-35 mer - Double Stranded

(19) United States
(12) Patent Application Publication
(10) Pub. No.: US 2017/0182205 A1
(43) Pub. Date: Jun. 29, 2017

(54) MEDICAL DEVICES WITH BIOFILM DISRUPTORS

(71) Applicant: ProclaRx LLC, Athens, OH (US)

(72) Inventors: Thomas J. ZUPANCIC, Columbus, OH (US); Joseph D. KITTLE, Athens, OH (US); Uday SANDBHOR, Columbus, OH (US); Richard S. BRODY, Columbus, OH (US)

(21) Appl. No.: 15/391,009

(22) Filed: Dec. 27, 2016

Related U.S. Application Data
(60) Provisional application No. 62/271,639, filed on Dec. 28, 2015.

(51) Int. Cl. A61L 15/46; A61L 27/54; A61L 27/34; A61L 27/28; A61L 15/20; A61L 15/28

(52) U.S. Cl. CPC A61L 15/46; A61L 15/20; A61L 15/28; A61L 27/34; A61L 27/28; A61L 27/54; A61L 2300/404; A61L 2420/00; A61L 2300/256; A61L 2300/258; A61L 2300/236

(57) ABSTRACT

A medical device for disrupting biofilms that includes a substrate; and at least one anti-biofilm composition bound to the substrate, wherein the at least one anti-biofilm composition is adapted to bind to DNA binding proteins present in a biofilm, and wherein the binding of the at least one anti-biofilm composition to the DNA binding protein disrupts the biofilm.

### CROSS-REFERENCE TO RELATED APPLICATIONS

This patent application claims the benefit of U.S. Provisional Patent Application Ser. No. 62/271,639 filed on Dec. 28, 2015 and entitled “Medical Devices with Biofilm Disruptors”, the disclosure of which is hereby incorporated by reference herein in its entirety and made part of the present U.S. utility patent application for all purposes.

### BACKGROUND OF THE INVENTION

The described invention relates in general to systems, methods, and devices for disrupting biofilms, and more specifically to medical devices such as bandages or implants that utilize, incorporate, or otherwise include one or more DNA-based compositions that act as disruptors of biofilms.

A biofilm is typically defined as any group of microorganisms wherein individual cells stick to each other on a substrate. These adherent cells are frequently embedded within a self-produced matrix of extracellular polymeric substance (EPS). Biofilm extracellular polymeric substance is a polymeric conglomeration generally composed of extracellular DNA, proteins, and polysaccharides. Biofilms may form on living or non-living surfaces and can be prevalent in natural, industrial and hospital settings. Microbial cells growing in a biofilm are physiologically distinct from planktonic cells of the same organism, which, by contrast, are single-cells that may float or swim in a liquid medium. Microbes form a biofilm in response to many factors, which may include cellular recognition of specific or non-specific attachment sites on a surface, nutritional cues, or in some cases, by exposure of planktonic cells to sub-inhibitory concentrations of antibiotics. When a cell switches to the biofilm mode of growth, it undergoes a phenotypic shift in behavior in which large suites of genes are differentially regulated.

Biofilms are involved in a wide variety of microbial infections in the body and biofilm development is an important step in the formation of many persistent and recurring bacterial infections. Infectious processes in which biofilms have been implicated include common problems such as bacterial vaginosis, urinary tract infections, catheter infections, middle-ear infections, formation of dental plaque, gingivitis, coating contact lenses, and less common but more lethal processes such as endocarditis, infections in cystic fibrosis, and infections of permanent indwelling devices such as joint prostheses and heart valves. Bacterial biofilms may impair cutaneous wound healing and reduce topical antibacterial efficiency in healing or treating infected skin wounds; thus, early detection and treatment of biofilms in wounds is crucial to successful chronic wound management.

Biofilms are assembled by communities of bacteria, often of diverse species, and as a consequence, bacteria can survive and accumulate in a contained environment distinct from free floating (planktonic) bacteria. Biofilms may contain polysaccharides and proteins as well as other molecules, but notably, pathogenic bacteria associated with disease seem to contain both DNA and a specific type of DNA binding protein (DNABII) as key components. The DNA has been shown to play a structural role beyond its well-known function as the genetic material of living organisms. The DNABII proteins, such as Hu and IHF, are bacterial proteins with no particularly close human counterparts. These DNA binding proteins have been reported to also play a pivotal role as molecules that bind at the intersection points of DNA, with the whole structure then taking on a net-like three-dimensional lattice configuration. In these instances, carbohydrates and other molecules trapped in the DNA-DNABII protein structure provide the rest of the biofilm bulk.

The importance of DNABII proteins in biofilm stability has been demonstrated by experiments performed on laboratory-grown bacterial biofilms as well as biofilms obtained from infected lungs. In bacteria, when a mutation is used to disrupt the function of DNABII, the ability of the bacteria to form a thick biofilm is lost. However, the ability to form the biofilm can be restored by the addition of DNABII proteins to the bacterial culture. Interestingly, antibodies that both bind DNABII proteins and block their binding to DNA can deplete these proteins from mature biofilms, thereby leading to disruption of the biofilm. By contrast, antibodies that bind to DNABII proteins, but that are not competitive with DNA binding are not effective in dissolving biofilms in vitro. In vivo animal studies in models of infectious disease demonstrate this same pattern, wherein antibodies that block binding of DNABII to its target DNA are effective in treating infections where non-blocking antibodies are not effective in clearing biofilms in animal models. Antibodies can be used to capture DNABII proteins from a biofilm without the need to penetrate the biofilm lattice itself. The binding proteins are in binding equilibrium with biofilm and it has been shown in vitro that DNABII proteins reversibly bind to a DNA target with nanomolar dissociation constants. Experiments demonstrating that (immersed in liquid) antibodies separated from the biofilm by a selective pore size membrane can remove DNABII proteins diffusing out of the biofilm, thereby leading to collapse of the biofilm.

Biofilms, which as described above, include DNA and DNABII proteins as critical components, are implicated in wounds susceptible to persistent or chronic bacterial infection, such as, for example burns, pressure sores and diabetic ulcers. Postoperative infections may also involve biofilms. Thus, there is an ongoing need for a wound dressing, surgical packing material, and medical devices generally that provide anti-biofilm properties.

### SUMMARY OF THE INVENTION

The following provides a summary of certain exemplary embodiments of the present invention. This summary is not an extensive overview and is not intended to identify key or critical aspects or elements of the present invention or to delineate its scope.

In accordance with one aspect of the present invention, a first medical device is provided. This medical device includes a substrate; and at least one anti-biofilm composition bound to the substrate, wherein the at least one anti-biofilm composition is adapted to bind to DNA binding proteins present in a biofilm, and wherein the binding of the at least one anti-biofilm composition to the DNA binding protein disrupts the biofilm.

In accordance with another aspect of the present invention, a second medical device is provided. This medical device includes a substrate; and at least one anti-biofilm composition bound to the substrate, wherein the at least one anti-biofilm composition is adapted to bind to DNA binding proteins present in a biofilm, wherein the DNA binding proteins include DNABII, and wherein the binding of the at least one anti-biofilm composition to DNABII disrupts the biofilm.

In yet another aspect of this invention, a third medical device is provided. This medical device includes a substrate, wherein the substrate is a bandage, wound dressing, or implant; and at least one anti-biofilm composition reversibly or releasably bound to the substrate, wherein the at least one anti-biofilm composition includes at least one DNA oligomer adapted to bind to DNA binding proteins present in a biofilm, wherein the DNA binding proteins include DNABII, and wherein the binding of at least one DNA oligomer to DNABII disrupts the biofilm.

Additional features and aspects of the present invention will become apparent to those of ordinary skill in the art upon reading and understanding the following detailed description of the exemplary embodiments. As will be appreciated by the skilled artisan, further embodiments of the invention are possible without departing from the scope and spirit of the invention. Accordingly, the drawings and associated descriptions are to be regarded as illustrative and not restrictive in nature.

### BRIEF DESCRIPTION OF THE DRAWINGS

The accompanying drawings, which are incorporated into and form a part of the specification, schematically illustrate one or more exemplary embodiments of the invention and, together with the general description given above and detailed description given below, serve to explain the principles of the invention, and wherein:

FIG. 1 is an illustration of the types of DNA structures synthesized and utilized in accordance with an exemplary embodiment of the present invention;

FIG. 2 is a photograph of a gel electrophoresis of the different DNA structures of the present invention and their respective molecular weights;

FIG. 3 is a graph depicting the binding of DNA oligomers labeled with biotin to a surface or substrate that has been coated with a DNABII protein, wherein DNA oligomers that bind to the surface are detected with streptavidin conjugated to horse radish peroxidase (HRP) followed by treatment with a HRP substrate (PS denotes DNA with two phosphorothioate diester linkages on both the 5′ and 3′ termini of an oligomer);

FIG. 4 is a graph illustrating the binding of a biotin labeled DNABII protein to immobilized DNA and the inhibition of this binding by the addition of a polyclonal antibody against the DNABII protein;

FIG. 5 is a bar graph illustrating the extent of biofilm disruption induced by the addition of a mixture of two DNA oligomers (PS-HJ+PS-Duplex), wherein the PS indicates the presence of terminal phosphorothioate linkages, and wherein the bar graph shows eight replicates for each condition; and

FIG. 6 is a bar graph illustrating the extent of biofilm inhibition induced by different concentrations of a diabody that bind to DNABII proteins, wherein the bar graph shows six replicates for each condition.

### DETAILED DESCRIPTION OF THE INVENTION

Exemplary embodiments of the present invention are now described with reference to the Figures. Although the following detailed description contains many specifics for purposes of illustration, a person of ordinary skill in the art will appreciate that many variations and alterations to the following details are within the scope of the invention. Accordingly, the following embodiments of the invention are set forth without any loss of generality to, and without imposing limitations upon, the claimed invention.

The present invention includes various exemplary devices that may be used as bandages for wound healing, in-dwelling implants (e.g., tubes, catheters, heart valves, etc.), or the like. This invention includes the following basic aspects or characteristics: (i) at least one DNA or antibody composition specific for at least one DNA binding protein found in a biofilm; (ii) an appropriate formulation or release mechanism for bringing the at least one anti-biofilm DNA or antibody composition into contact with the at least one DNA binding protein; (iii) appropriate distribution of the at least one DNA or antibody composition within or on a device such as a bandage or implant; (iv) appropriate persistence and/or clearance of the at least one DNA or antibody composition (e.g., stability if the DNA composition is in a desired configuration); and (v) an appropriate substrate, matrix, or encapsulation means for the at least one DNA or antibody composition.

The efficacy or effectiveness of the present invention involves an appropriate and functional interaction between the elements listed in the previous paragraph. The specific composition and structure of the at least one DNA composition or antibody determines the biofilm disruption capacity (i.e., specific required dose). The release, persistence, and clearance of the at least one DNA composition or antibody is carefully modulated to produce desired therapeutic effects. The formulation and loading of the at least one DNA composition or antibody into on onto the device is specifically designed to achieve multiple properties and characteristics important for achieving efficacy, including: (a) stability of the DNA or antibody; (b) proper release and distribution to the site of application (this result is also a provided by the design of the device); and (c) appropriate therapeutic dosing over an appropriate time (a combination/careful balance of multiple elements from list above).

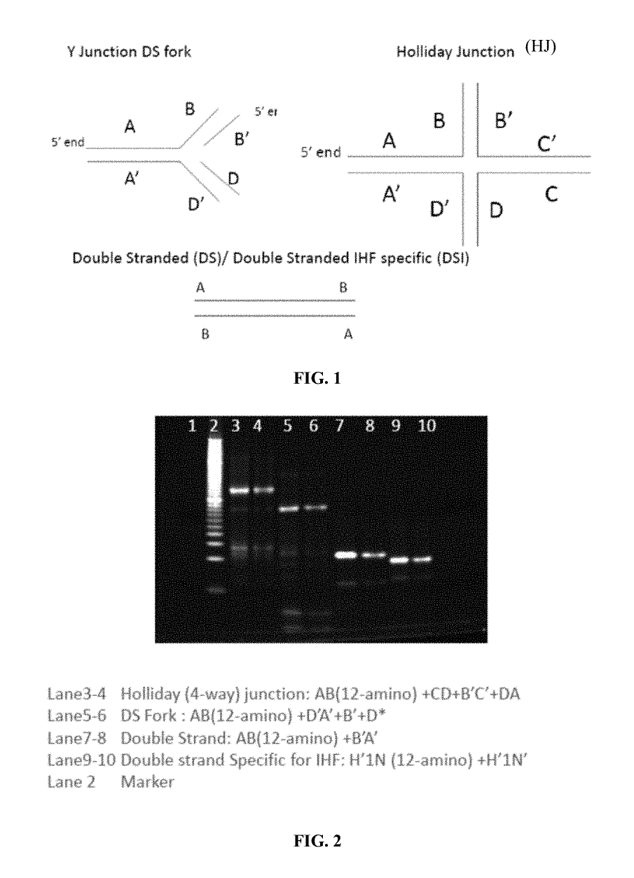

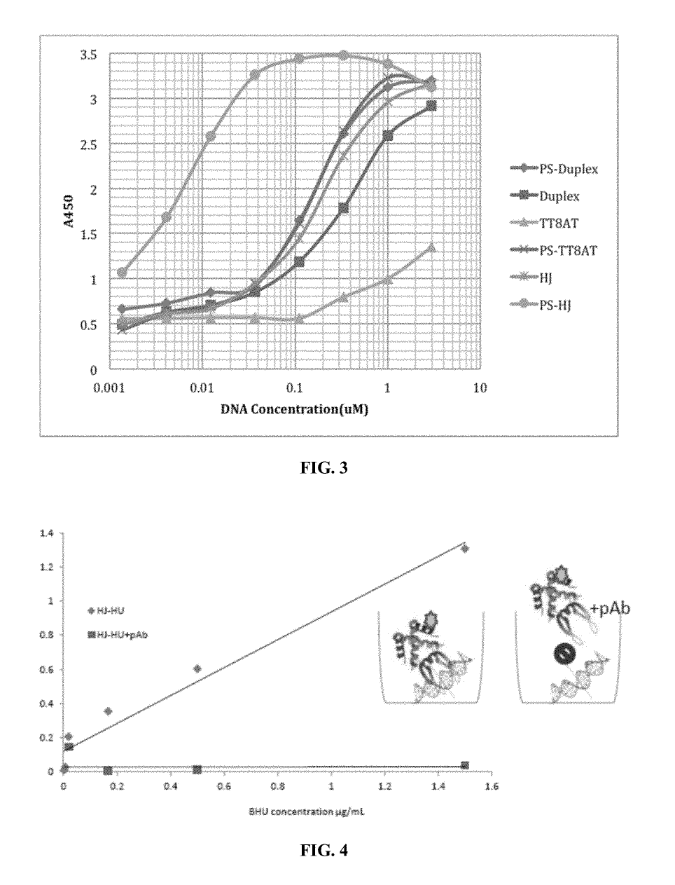

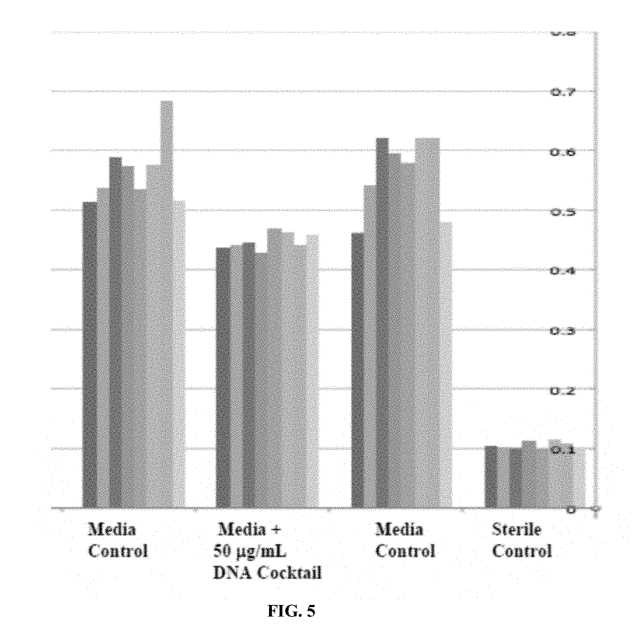

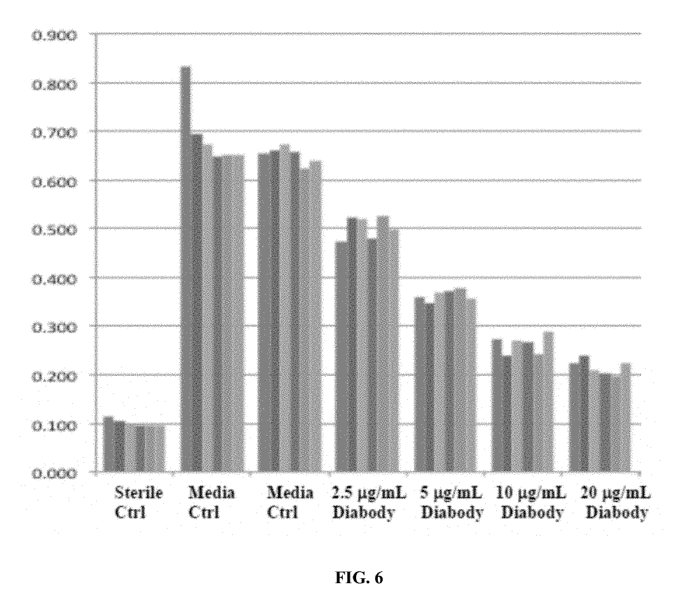

With reference to FIGS. 1-6, important aspects of the at least one anti-biofilm DNA composition of this invention include: (i) structure, further including: (a) a replication fork; (b) a Holliday junction; and (c) mismatched DNA/gapped DNA; and (ii) base composition (potential factors), further including: (a) A-T/G-C content; (b) DNA versus Z DNA, etc. (c) distribution of A-T vs G-C (e.g., poly A stretch); (d) asymmetric distribution of purines and prymidines; (e) tethered/blocked DNA ends; and (f) supercoiled/circular structures.

SEQ ID NOS: 1-13 (see also TABLES 1-2, below) provide examples of oligonucleotides used to prepare the DNA structures incorporated into certain embodiments of the present invention. SEQ ID NOS: 1-7 are DNA oligomer components of the structures shown in FIG. 1 (5′→3′). SEQ ID NOS: 8-13 are double stranded oligomers (complementary oligomers with gaps and mismatches; 5′→3′). Structures such as those shown in SEQ ID NOS: 1-13 and TABLES 1-2, are known to bind to DNABII proteins with nanomolar or lower dissociation constants (see, for example, Kamashev, D. and Rouviere-Yaniv, J. (2000) “The histone-like protein Hu binds specifically to DNA recombination and repair intermediates” The EMBO Journal 19, 6527-6535; Tjokro, N. O. et al. (2014) “A Biochemical Analysis of the Interaction of Porphyromonas gingivalis Hu PG0121 Protein with DNA” PLOS ONE 9, 1-12; Swinger, K. K. and Rice, P. A. (2007) “Structure-Based Analysis of Hu-DNA binding” J. Mol. Biol. 365, 1005-1016; and Vivas, P. et al. (2012) “Mapping the Transition State for DNA Bending by IHF” J. Mol. Biol. 418, 300-315. The DNA structures shown in SEQ ID NOS: 1-13 and TABLES 1-2 can be made either with normal phospho-diester bonds or with phosphorothioate-diester bonds to inhibit DNA degradation catalyzed by bacterial nucleases (see, Clafre, S. A. et al. (1995) “Stability and functional effectiveness of phosphorothioate modified duplex DNA and synthetic mini-genes” Nucleic Acids Research 23, 4134-4142.

TABLE 1
DNA Oligomer Components of the Structures Shown in FIG. 1
(5′ → 3′)

| | | |
|---|---|---|
| SEQ ID NO: 1 | AB-40 mer | GGAACCTTGG CCTTAACCAA CCAAGGTTCC GGTTAAGGAA |
| SEQ ID NO: 2 | CD-41 mer | GCAACGTGTG CCGTTAACGA ACCTAGGATG GGCATTAGGT A |
| SEQ ID NO: 3 | B′C′-41 mer | TTCCTTAACC GGAACCTTGG TTCGTTAACG GCACACGTTG C |
| SEQ ID NO: 4 | D′A′-40 mer | TACCTAATGC CCATCCTAGG TTGGTTAAGG CCAAGGTTCC |
| SEQ ID NO: 5 | B′A′-40 mer | TTCCTTAACC GGAACCTTGG TTGGTTAAGG CCAAGGTTCC |
| SEQ ID NO: 6 | B′-20 mer | TTCCTTAACC GGAACCTTGG |
| SEQ ID NO: 7 | D-20 mer | CCTAGGATGG GCATTAGGTA |

TABLE 2
Double Stranded Oligomers
(complementary oligomers with gaps and mismatches; 5′ → 3′)

| | | |
|---|---|---|
| SEQ ID NO: 8 | H′1N-35 mer | GGCCAAAAAA GCATTGCTTA TCAATTTGTT GCACC |
| SEQ ID NO: 9 | H′1N′-35 mer | CGGTGCAACA AATTGATAAG CAATGCTTTT TTGGC |
| SEQ ID NO: 10 | Duplex 1-44 mer | TACGTTTGTT GCATGCTTAC AAATTGTTGC AACGTTGTTT TACG |
| SEQ ID NO: 11 | Duplex 1′-44 mer | CGTAAAACAA CGTTGCTTAC AATTTGTTGC ATGCAACAAA CGTA |
| SEQ ID NO: 12 | TT8AT-36 mer | CGGTGCAACA ATATGATAAG CTTTGCTTTT TTGGCC |
| SEQ ID NO: 13 | H′1N-35 mer | GGCCAAAAAA GCATTGCTTA TCAATTTGTT GCACC |

The present invention includes unique properties based on the incorporation of components specifically designed and optimized for adsorbing DNABII binding proteins from infected wounds. While anti-DNABII binding protein molecules are currently under development for use as therapeutics, incorporation thereof into a device is novel in that the binding molecules are not applied as a therapeutic, but as a means to make the device itself capable of dispersing biofilms. In particular, the device may reduce the exposure of the wound directly to the anti-biofilm agent and simplify its removal or replenishment as the wound treatment proceeds. This invention includes embodiments wherein anti-biofilm agents are initially embedded in a bandage or device and are then released in a controlled manner to interact with and ultimately disrupt a biofilm that has formed at a wound site or implantation site. This invention also includes embodiments wherein anti-biofilm agents remain bound to a bandage or device and are operative to capture DNA binding proteins such as DNABII that diffuse out of a wound site. Capturing DNA binding proteins in this manner is then operative to disrupt a target biofilm. The methods disclosed in U.S. Pat. No. 8,999,291 are relevant to certain embodiments of this invention and U.S. Pat. No. 8,999,291 is incorporated by reference herein in its entirety and made part of this disclosure for all purposes. As will be appreciated by one of ordinary skill in the art, a biological molecule that disrupt biofilms in vitro has been shown to be effective, as part of a wound care system, in treating biofilm containing chronic wounds in an animal model. In this case, the biological molecule is an enzyme that depolymerizes a polysaccharide needed for biofilm formation (see, Gawande, P. V. et al. (2011) “In Vitro Antimicrobial and Antibiofilm Activity of DispersinB®-Triclosan Wound Gel against Chronic Wound-associated Bacteria” The Open Antimicrobial Journal 3, 12-16; and Gawande, P. V. et al. (2014) “Antibiofilm Efficacy of DispersinB Wound Spray Used in Combination with a Silver Wound Dressing” Microbiology Insights 7, 9-13.

In one embodiment, the device of the present invention is a simple cotton gauze that is coated with molecules that have affinity for DNA binding proteins such as the DNA oligomers described in TABLES 1-2. Another example of one such molecule is heparin, having a known affinity for positively charged proteins, but with specific applications in the chromatographic separation/purification of polynucleotide binding proteins. Attaching molecules such as heparin to a bandage material such as cotton provides a stable matrix for capturing DNABII proteins when the material is brought into contact with a wound. Diffusion of DNABII proteins out of the biofilm then results in the collapse of the biofilm that shelters the pathogenic bacteria.

Due to its poly-anionic carbohydrate structure, heparin has the ability to bind many types of polynucleotide binding proteins. DNABII proteins are known to bind to heparin as demonstrated by its use as a component of affinity binding resins for the capture and purification of such proteins. Thus it is capable of anti-biofilm activity. Heparin included in the device of this invention may be attached in a manner that reduces or eliminates its ability to diffuse into the wound site itself. Heparin may be expected to prevent immediate clotting of fibrogen in the wound, keeping the wound moist while still protected by the wound dressing, thereby facilitating cellular repair of the wound even as the biofilm burden is reduced and maintained.

Specific DNABII binding matrices can also be engineered by coating wound care materials with other molecules that are selective for DNABII binding proteins. One type of biomolecule is a DNABII-specific antibody which blocks binding of the DNA matrix to the DNABII protein (see FIG. 4). Such antibodies disrupt biofilms by coating a device with an effective antibody and the device preferentially adsorbs DNABII proteins when brought in contact or in equilibrium with a biofilm. Over time, this wound care device sequesters DNABII protein to an extent that the biofilm is disrupted and the bacteria are exposed to the body's own defenses. In addition, the wound device may simultaneously deliver antibiotic to the wound site to speed the eradication of the target pathogen or pathogens. Polyclonal and monoclonal antibodies have been prepared by immunizing animals with DNABII proteins or DNABII peptide fragments. These antibodies bind to DNABII proteins and disrupt biofilms in in-vivo experiments. These antibodies have also been shown to reduce or eliminate bacterial infections in animal models of otitis media (Haemophilus influenza) and lung infection (Pseudomonas aeruginosa). See, U.S. Pat. No. 8,999,291 and Novotny, L. A. (2016) “Monoclonal antibodies against DNA-binding tips of DNABII proteins disrupt biofilms in vitro and induced bacterial clearance in vivo” EBioMedicine 10, 33-44.

Protein domains of antibodies that are the specific binding domains that capture DNABII have been identified. These so called hypervariable binding domains have been used to engineer smaller proteins and peptides that retain binding specificity to the target antigen. One example is a so called diabody consisting of an antibody heavy and light chain expressed as a single peptide (for example in bacteria) and dimerized to form a bivalent molecule or diabody with affinity for DNABII protein. These and other molecules designed as binding proteins using domains originally identified in antibodies are collectively known as “antibody fragment proteins”. Such proteins may have cost and stability advantages over the use of whole antibodies as coatings in anti-biofilm wound devices.

Use of DNA oligonucleotides as DNABII binding molecules in a wound care device is an aspect of this invention. DNA is the natural target for DNABII binding proteins. Binding of DNA to a specific DNABII protein depends on an “indirect” read of the DNA sequence at the appropriate binding site. A DNA helix normally appears as a somewhat rigid rod, while certain DNA sequences can lend increased flexibility to this rod and in some cases may result in the formation of a permanent bend in the DNA molecule.

DNA base pairing (by insertions/deletions or mutations, for example) can also cause the DNA structure to kink. For Hu proteins, the protein binds to DNA and itself induces a bend in the DNA molecule. This occurs as protein residues bind to the minor groove of the DNA and the DNA itself also wraps around the protein to maximize contacts of the negatively charged backbone of the molecule to positive residues on the sides of the protein. Thus, not all DNA binds equally well to a given HU binding protein with flexible or bent DNA typically binding with a higher affinity. Certain DNA structures that include bent DNA, including replication forks and Holiday (DNA recombination) junctions, may also have high affinity for binding proteins (see FIG. 1).

A second relevant factor in using DNA to capture binding protein is the natural role of DNA in the biofilm. Exogenous DNA of regular molecular weight added to a culture can be readily incorporated into a biofilm. However, examination of the biofilm reveals that the DNAB lattice consists of long, intersecting DNA strands with the binding proteins at clearly separated nodes where strand appear to intersect. Thus, short oligonucleotides (e.g. 10, 20, 30 or 40 bases of double or single stranded DNA) cannot act to bring together separate portions of the biofilm and the DNA would be associated with only one or two DNABII proteins. Properly designed DNA molecules, therefore, by themselves, would not form a stable biofilm lattice. In addition, because of their relative size, smaller high affinity DNA molecules may have greater high affinity per unit mass than high molecular weight DNA derived from genomic DNA, with its more average DNA composition and far fewer sites per mass unit.

One embodiment of this invention uses DNA oligonucleotides engineered or selected for high affinity to DNABII proteins to coat the wound dressing. The DNABII proteins in the target biofilm diffuse to the DNA oligo incorporated into the bandage, where they would be unavailable for biofilm formation. Such sequestration of the DNABII proteins would then reduce the burden of biofilm in the wound.

Each of the biofilm disruptive agents of this invention can be used in combination with each other and with other wound healing agents such as antibiotics or barrier salves. Biofilm disruptive agents (alone or in combination with other compositions) can also be incorporated into creams or gels that include porous plastics, hydrogels, alginates, liposomes, or mesoporous silica nanoparticles. In addition, biofilm disruptive agents may be incorporated in to biodegradable materials such as poly(lactic-co-glycolic) acid (PLGA) for slowly bringing a steady supply of available binding sites in contact with a wound. An example of such an application might be a suture or mesh that contains at least one anti-biofilm agent. Biofilm disruptive material can be fabricated into more complex devices such as tympanic membrane tubes used to treat otitis media (i.e., middle ear infection) or in medical stents or catheters.

While the present invention has been illustrated by the description of exemplary embodiments thereof, and while the embodiments have been described in certain detail, it is not the intention to restrict or in any way limit the scope of the appended claims to such detail. Additional advantages and modifications will readily appear to those skilled in the art. Therefore, the invention in its broader aspects is not limited to any of the specific details, representative devices and methods, and/or illustrative examples shown and described. Accordingly, departures may be made from such details without departing from the spirit or scope of the general inventive concept.

What is claimed:

1. A medical device for disrupting biofilms, comprising:
(a) a substrate; and
(b) at least one anti-biofilm composition bound to the substrate, wherein the at least one anti-biofilm composition is adapted to bind to DNA binding proteins present in a biofilm, and wherein the binding of the at least one anti-biofilm composition to the DNA binding protein disrupts the biofilm.

2. The medical device of claim 1, wherein the substrate is a bandage or wound dressing.

3. The medical device of claim 1, wherein the substrate is an implant.

4. The medical device of claim 1, wherein the at least one anti-biofilm composition is either releasably bound to the substrate or permanently bound to the substrate.

5. The medical device of claim 1, wherein the at least one anti-biofilm composition is releasably bound to the substrate, and wherein the at least one anti-biofilm composition is formulated into a cream or gel prior to substrate binding.

6. The medical device of claim 1, wherein the at least one anti-biofilm composition includes heparin.

7. The medical device of claim 1, wherein the at least one anti-biofilm composition includes at least one DNA oligomer.

8. The medical device of claim 7, where in the at least one DNA oligomer is selected from the group consisting of SEQ ID NOS. 1-7.

9. The medical device of claim 7, where in the at least one DNA oligomer is selected from the group consisting of SEQ ID NOS. 8-13.

10. The medical device of claim 1, wherein the DNA binding proteins include DNABII

11. The medical device of claim 10, wherein the at least one anti-biofilm composition includes at least one antibody specific to DNABII, at least one diabody with affinity for DNABII, at least one DNA oligomer that binds to DNABII, or a combination thereof.

12. A medical device for disrupting biofilms, comprising:
(a) a substrate; and
(b) at least one anti-biofilm composition bound to the substrate, wherein the at least one anti-biofilm composition is adapted to bind to DNA binding proteins present in a biofilm, wherein the DNA binding proteins include DNABII, and wherein the binding of the at least one anti-biofilm composition to DNABII disrupts the biofilm.

13. The medical device of claim 12, wherein the substrate is a bandage, wound dressing, or implant.

14. The medical device of claim 12 wherein the at least one anti-biofilm composition is either releasably bound to the substrate or permanently bound to the substrate.

15. The medical device of claim 12, wherein the at least one anti-biofilm composition is releasably bound to the substrate, and wherein the at least one anti-biofilm composition is formulated into a cream or gel prior to substrate binding.

16. The medical device of claim 12, wherein the at least one anti-biofilm composition includes at least one antibody specific to DNABII, at least one diabody with affinity for DNABII, at least one DNA oligomer that binds to DNABII, or a combination thereof.

17. The medical device of claim 12, wherein the at least one anti-biofilm composition is a DNA oligomer selected from the group consisting of SEQ ID NOS. 1-7.

18. The medical device of claim 12, wherein the at least one anti-biofilm composition is a DNA oligomer selected from the group consisting of SEQ ID NOS. 8-13.

19. A medical device for disrupting biofilms, comprising:
(a) a substrate, wherein the substrate is a bandage, wound dressing, or implant; and
(b) at least one anti-biofilm composition releasably bound to the substrate, wherein the at least one anti-biofilm composition includes at least one DNA oligomer adapted to bind to DNA binding proteins present in a biofilm, wherein the DNA binding proteins include DNABII, and wherein the binding of at least one DNA oligomer to DNABII disrupts the biofilm.

20. The medical device of claim 19, where in the at least one DNA oligomer is selected from the group consisting of SEQ ID NOS. 1-13.
